(19) United States
(12) Patent Application Publication
(10) Pub. No.: US 2026/0314944 A1
(43) Pub. Date: Oct. 15, 2026

(54) BIOLOGICAL SIGNAL SENSOR MODULE

(71) Applicant: Murata Manufacturing Co., Ltd., Nagaokakyo-shi (JP)

(72) Inventors: Kenichi MORI, Nagaokakyo-shi (JP); Masanori MORITA, Nagaokakyo-shi (JP); Toshio TSUJI, Higashi-Hiroshima-shi (JP)

(21) Appl. No.: 19/463,696

(22) Filed: Jan. 29, 2026

Related U.S. Application Data
(63) Continuation of application No. PCT/JP2024/025948, filed on Jul. 19, 2024.

(30) Foreign Application Priority Data
Aug. 2, 2023 (JP) 2023-125973

(51) Int. Cl. A61B 5/024; A61B 5/00

(52) U.S. Cl. CPC A61B 5/024; A61B 5/721; A61B 5/7225

(57) ABSTRACT

A biological signal sensor module is provided that includes one or more sensors having a piezoelectric film and outputting a first output and a second output due to deformation of a skin surface, a detection circuit, and a signal processing unit. The detection circuit amplifies the first output to output a first signal including a biological signal and a body motion signal, and the detection circuit amplifies the second output to output a second signal including a body motion signal. The signal processing unit estimates a biological signal based on the first signal and the second signal by using a preset biological signal estimation algorithm.

### CROSS REFERENCE TO RELATED APPLICATIONS

This application is a continuation of PCT Application No. PCT/JP2024/025948, filed Jul. 19, 2024, which claims priority to Japanese Patent Application No. 2023-125973, filed Aug. 02, 2023, the entire contents of each of which are hereby incorporated by reference in their entireties.

### TECHNICAL FIELD

The present disclosure relates to a biological signal sensor module for measuring a biological signal.

### BACKGROUND

Japanese Patent Application Laid-Open No. 2010-172645 (hereinafter “Patent Document 1”) discloses a heartbeat detection device that detects a heartbeat signal derived from a heartbeat of a human body. The heartbeat detection device includes a first sensor, a second sensor, and a filtering unit. The first sensor outputs a signal in which a heartbeat signal and a noise signal are mixed. The second sensor outputs a body motion signal accompanying body motion of a human body. The filtering unit generates an adaptive filter based on a body motion signal, extracts a noise signal in a signal in which a heartbeat signal and a noise signal are mixed, and removes the noise signal from the signal in which a heartbeat signal and a noise signal are mixed.

In the heartbeat detection device disclosed in Patent Document 1, an acceleration sensor is employed as the second sensor that outputs a body motion signal. However, although the acceleration sensor can measure movement of a wearing site, the acceleration sensor cannot accurately measure deformation of the surface of the skin to be measured for a pulse wave. Therefore, when a noise signal in a signal in which a heartbeat signal and a noise signal are mixed is removed based on a body motion signal from the acceleration sensor, pulse measurement can be performed, but accuracy of pulse wave measurement may be deteriorated.

Furthermore, in this heartbeat detection device, application to measurement of a biological signal other than a heartbeat signal is not assumed.

### SUMMARY OF THE INVENTION

In view of the foregoing, the present disclosure provides a biological signal sensor module that accurately measures a biological signal even when there is body motion.

In an exemplary aspect, a biological signal sensor module includes one or more sensors that have a piezoelectric film and that outputs a first output and a second output due to deformation of a skin surface, a detection circuit, and a signal processing unit. The detection circuit amplifies the first output to output a first signal including a biological signal and a body motion signal, the detection circuit amplifies the second output to output a second signal including a body motion signal, and the signal processing unit estimates a biological signal based on the first signal and the second signal by using a preset biological signal estimation algorithm.

According to the exemplary aspects of the present disclosure, a biological signal can be accurately measured even when there is body motion.

### BRIEF DESCRIPTION OF THE DRAWINGS

FIG. 1 is a block diagram illustrating a biological signal sensor module 1 according to an exemplary aspect.

FIG. 2(A) is an external perspective view illustrating a biological signal sensor 11 according to an exemplary aspect. FIG. 2(B) is a sectional view illustrating the biological signal sensor 11.

FIG. 3 is a plan view illustrating a piezoelectric film 21 according to an exemplary aspect.

FIG. 4 is a conceptual diagram illustrating arrangement of the biological signal sensor 11 and a body motion sensor 12 when the biological signal sensor module 1 is used according to an exemplary aspect.

FIG. 5 is a diagram illustrating an example of a mixed signal, a body motion signal, and a pulse wave signal used for setting a biological signal estimation algorithm according to an exemplary aspect.

FIG. 6 is a diagram illustrating an example of a mixed signal and a pulse wave signal estimated using an LSTM according to an exemplary aspect.

FIG. 7 is a diagram illustrating an example of a mixed signal and a pulse wave signal estimated using a bidirectional LSTM according to an exemplary aspect.

FIG. 8 is a block diagram illustrating a biological signal sensor module 1a according to an exemplary aspect.

FIG. 9 is a sectional view illustrating the biological signal sensor 11 and the body motion sensor 12 according to an exemplary aspect.

FIG. 10 is a block diagram illustrating a biological signal sensor module 1b according to an exemplary aspect.

FIG. 11 is a sectional view illustrating a biological signal sensor 11b and a body motion sensor 12b according to an exemplary aspect.

FIG. 12 is a plan view illustrating a signal electrode 24b according to an exemplary aspect.

FIG. 13 is a block diagram illustrating a biological signal sensor module 1c according to an exemplary aspect.

### DETAILED DESCRIPTION

Hereinafter, a plurality of exemplary embodiments of the present disclosure will be described. In each embodiment, a difference from description before the embodiment will be mainly described. The same function and effect by the same configuration will not be mentioned for each embodiment.

### Exemplary Embodiment

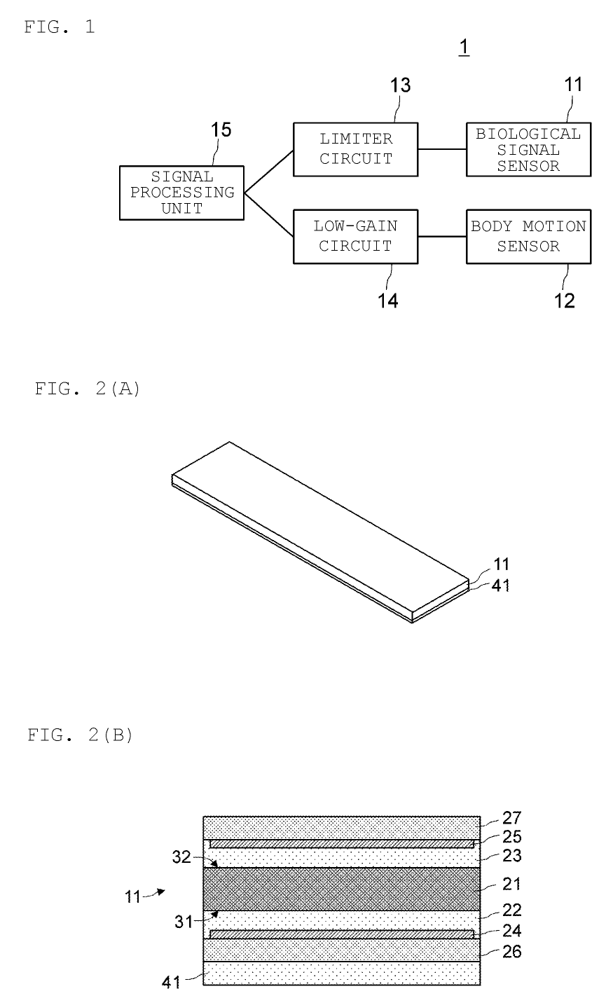

FIG. 1 is a block diagram illustrating a biological signal sensor module 1 in accordance with an exemplary aspect. In operation, the biological signal sensor module 1 estimates a biological signal, such as a pulse wave signal, as one example (e.g., of a user or patient). The biological signal sensor module 1 includes a biological signal sensor 11, a body motion sensor 12, a limiter circuit 13, a low-gain circuit 14, and a signal processing unit 15. The biological signal sensor 11 outputs a signal, which can be considered a first output or a first output signal, due to deformation of a skin surface. The body motion sensor 12 outputs a signal, which can be considered a second output or a second output signal, due to deformation of a skin surface. In the exemplary aspect, the biological signal sensor 11 and the body motion sensor 12 include a piezoelectric film. The limiter circuit 13 amplifies the output of the biological signal sensor 11 to output a mixed signal, which can be considered a first signal, including a pulse wave signal and a body motion signal. The low-gain circuit 14 amplifies output of the body motion sensor 12 to output a body motion signal, which can be considered a second signal. In an exemplary aspect, the limiter circuit 13 and the low-gain circuit 14 form a detection circuit that outputs a mixed signal and a body motion signal. The signal processing unit 15 estimates a pulse wave signal based on a mixed signal and a body motion signal by using a preset biological signal estimation algorithm that can be stored in memory.

In an exemplary aspect, the limiter circuit 13 is adjusted in gain so as to appropriately amplify a small sensor output corresponding to a small displacement, such as displacement of a skin surface due to a biological signal (e.g., a pulse wave accompanying a heartbeat). On the other hand, the limiter circuit 13 decreases gain with respect to a relatively large sensor output corresponding to a relatively large displacement of a skin surface, such as displacement of a skin surface due to body motion (e.g., a muscular movement of the user’s body). By this configuration, the limiter circuit 13 outputs a signal that can be processed by the signal processing unit 15 without saturating its output. In this way, the limiter circuit 13 detects deformation of a skin surface due to a pulse wave and body motion and outputs a mixed signal.

The low-gain circuit 14 is adjusted in gain to appropriately amplify a relatively large sensor output corresponding to a relatively large displacement, such as displacement of a skin surface due to body motion, and has a relatively low gain. In this manner, the low-gain circuit 14 does not saturate its output, detects deformation of a skin surface due to body motion, and outputs a body motion signal.

The signal processing unit 15 includes a CPU (computer processing unit), a memory, and the like, stores a program in which a biological signal estimation algorithm is mounted, and estimates a pulse wave signal by executing the program. In other words, the CPU is configured to execute the algorithms and calculations described herein by executing the program (e.g., code) stored on the memory as would be appreciated to one skilled in the art.

FIG. 2(A) is an external perspective view illustrating the biological signal sensor 11. FIG. 2(B) is a sectional view illustrating the biological signal sensor 11. As illustrated in FIG. 2(A), the biological signal sensor 11 is of a film type and has an elongated shape. As illustrated in FIG. 2(B), the biological signal sensor 11 includes a piezoelectric film 21, adhesive materials 22 and 23, a signal electrode 24, a reference electrode 25, and flexible substrates 26 and 27. The piezoelectric film 21 has a first main surface 31 and a second main surface 32 opposite to the first main surface 31.

The signal electrode 24 is formed on (e.g., disposed on) the flexible substrate 26, and is attached to the first main surface 31 of the piezoelectric film 21 by the adhesive material 22. The reference electrode 25 is formed on (e.g., disposed on) the flexible substrate 27, and is attached to the second main surface 32 of the piezoelectric film 21 by the adhesive material 23. The signal electrode 24 is disposed so as to face the reference electrode 25 across the piezoelectric film 21.

The biological signal sensor module 1 further includes an adhesive material 41 disposed on a main surface of the biological signal sensor 11.

In a modification example, the biological signal sensor module may include another material or mechanism instead of the adhesive material 41 in order to fix the biological signal sensor 11 to skin at the time of use as would be appreciated to one skilled in the art.

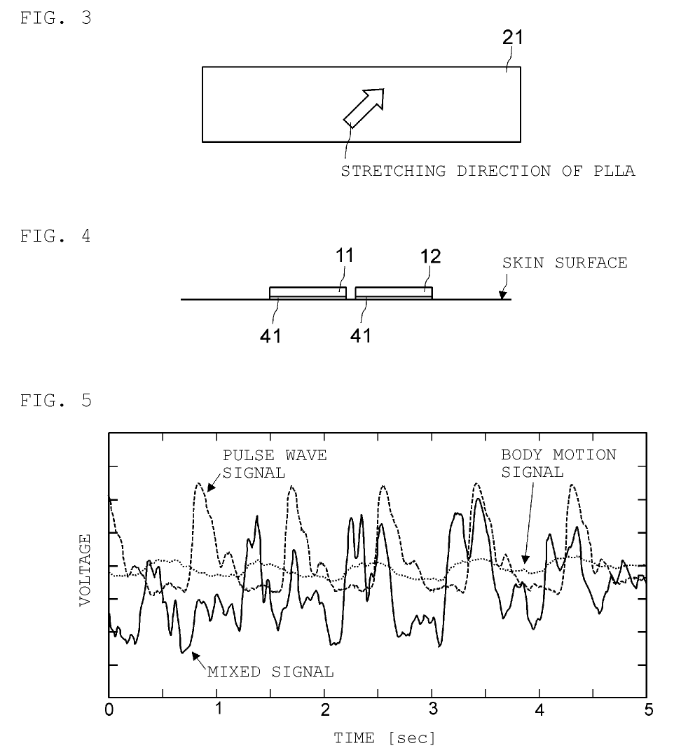

FIG. 3 is a plan view illustrating the piezoelectric film 21. The piezoelectric film 21 is formed from uniaxially stretched poly-L-lactic acid (PLLA). A stretching direction of PLLA of the piezoelectric film 21 forms an angle of 45° with respect to a longitudinal direction of the piezoelectric film 21, that is, a longitudinal direction of the biological signal sensor 11. By this configuration, deformation in a thickness direction of the biological signal sensor 11 can be detected with high sensitivity.

In a modification example, the piezoelectric film 21 may be formed from another piezoelectric material instead of PLLA.

In an exemplary aspect, the body motion sensor 12 has the same structure as the biological signal sensor 11, and the adhesive material 41 is disposed in the same manner as the biological signal sensor 11. For this reason, illustration and description of the structure of the body motion sensor 12 are omitted.

The signal electrode 24 and the reference electrode 25 of the biological signal sensor 11 are connected to the limiter circuit 13, and the signal electrode 24 and the reference electrode 25 of the body motion sensor 12 are connected to the low-gain circuit 14.

In a modification example, the biological signal sensor 11 and the body motion sensor 12 may have alternative shapes instead of an elongated shape. Further, the biological signal sensor 11 and the body motion sensor 12 may have different shapes or sizes from each other in an exemplary aspect.

FIG. 4 is a conceptual diagram illustrating arrangement of the biological signal sensor 11 and the body motion sensor 12 when the biological signal sensor module 1 is used. When the biological signal sensor module 1 is used, the biological signal sensor 11 and the body motion sensor 12 are disposed so as to be close to each other and are attached to a skin surface by the adhesive material 41. When a skin surface is deformed in a direction perpendicular to the skin surface due to propagation of a pulse wave accompanying a heartbeat, the piezoelectric film 21 in each sensor is deformed in its thickness direction and generates polarization charges. The limiter circuit 13 and the low-gain circuit 14 convert the polarization charges into voltage and amplify the voltage with a predetermined gain.

In an exemplary aspect, the biological signal estimation algorithm is an LSTM *Long Short-Term Memory) neural network. The biological signal estimation algorithm is set by machine learning using a mixed signal, a body motion signal, and a pulse wave signal as training data. That is, the biological signal estimation algorithm is set by machine learning using a mixed signal and a body motion signal as input data and a pulse wave signal as target output data.

In a modification example, the biological signal estimation algorithm may be a neural network different from an LSTM or may be an algorithm different from a neural network.

FIG. 5 is a diagram illustrating an example of a mixed signal, a body motion signal, and a pulse wave signal used for setting the biological signal estimation algorithm. A mixed signal has an irregular waveform resulting from disturbance, by body motion, of deformation of a skin surface due to a pulse wave.

In order to prepare training data (which can be stored in the memory), for example, the biological signal

sensor 11 and the body motion sensor 12 are arranged on the right wrist, and another one of the biological signal sensor 11 is arranged on the left wrist. By moving the right hand and resting the left hand at the same time, a mixed signal, a body motion signal, and a pulse wave signal can be acquired from output of the limiter circuit 13 connected to the biological signal sensor 11, output of the low-gain circuit 14 connected to the body motion sensor 12, and output of another one of the limiter circuit 13 connected to the other biological signal sensor 11.

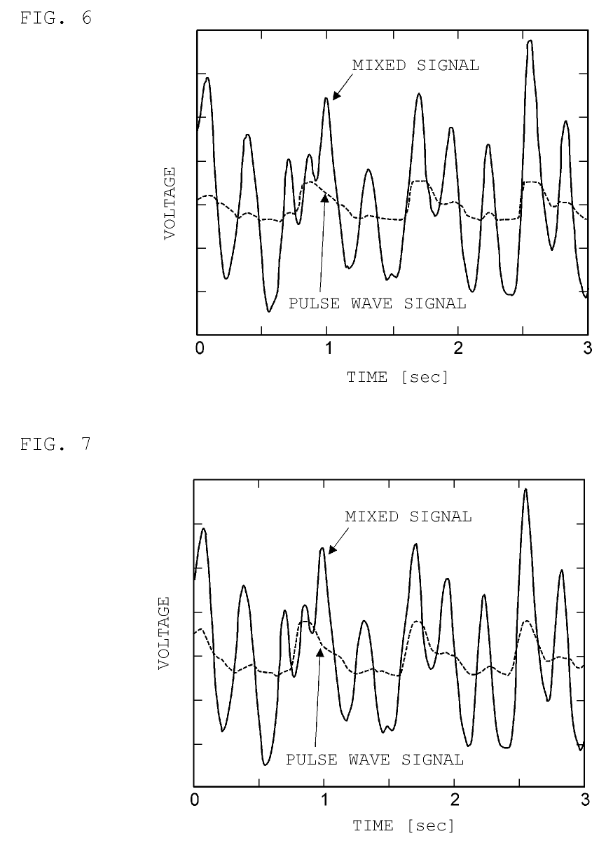

FIG. 6 is a diagram illustrating an example of a mixed signal and a pulse wave signal estimated using an LSTM. In a waveform of the mixed signal, peaks appear randomly, and it is not possible to determine which peak occurs according to a pulse wave. On the other hand, in a waveform of the estimated pulse wave signal, peaks appear at intervals of about 1 second, and thus a pulse wave can be measured.

According to the present embodiment, since the biological signal sensor 11 and the body motion sensor 12 are directly attached to a skin surface, the biological signal sensor 11 and the body motion sensor 12 have excellent sensitivity to deformation of the skin surface.

Further, first, as described above, the biological signal sensor 11 and the body motion sensor 12 accurately capture deformation of a skin surface. Second, the respective outputs of the biological signal sensor 11 and the body motion sensor 12 are generated by a piezoelectric film. From these two points, since a mixed signal and a body motion signal have a relatively simple relationship as input data of the biological signal estimation algorithm, accuracy of estimating a pulse wave signal is improved. Therefore, a pulse wave signal can be accurately measured even in the presence of body motion.

Furthermore, in an LSTM, a target waveform is estimated by strongly inheriting influence of an input waveform immediately before with respect to input of waveform data that is time-series data. For this reason, in an LSTM, a temporally short regular waveform, such as a waveform of a biological signal, such as a pulse wave signal, can be accurately estimated. Therefore, accuracy of estimating a pulse wave signal is further improved. Therefore, a pulse wave signal can be further accurately measured even in the presence of body motion.

Further, the biological signal sensor 11 and the body motion sensor 12 are of a film type. For this reason, even if the biological signal sensor 11 and the body motion sensor 12 are worn when the biological signal sensor module 1 is used, user's discomfort is suppressed and/or minimized.

### First modification example

According to a present modification example, the biological signal sensor module differs from the biological signal sensor module 1 in that the biological signal estimation algorithm is a bidirectional LSTM neural network.

FIG. 7 is a diagram illustrating an example of a mixed signal and a pulse wave signal estimated using a bidirectional LSTM. In a waveform of the mixed signal, peaks appear randomly, and it is not possible to determine which peak occurs according to a pulse wave. On the other hand, in a waveform of the estimated pulse wave signal, peaks appear at intervals of about 1 second, and a pulse wave can be measured. Furthermore, in a case of a bidirectional LSTM, a peak is clearer than that in a case of an LSTM.

Furthermore, in a bidirectional LSTM, a target waveform is estimated in consideration of an input waveform immediately after in addition to an input waveform immediately before with respect to input of waveform data that is time-series data. For this reason, according to the present modification example, accuracy of estimating a pulse wave signal is further improved. Therefore, a pulse wave signal can be measured with higher accuracy even in the presence of body motion.

### Second modification example

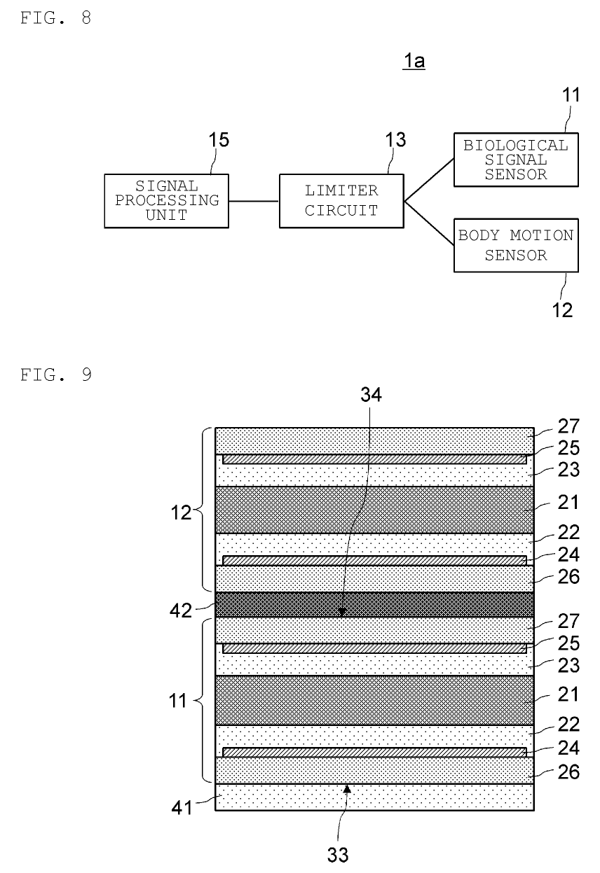

FIG. 8 is a block diagram illustrating a biological signal sensor module 1a. FIG. 9 is a sectional view illustrating the biological signal sensor 11 and the body motion sensor 12. The biological signal sensor module 1a is different from the biological signal sensor module 1 in the following point.

As illustrated in FIG. 9, the biological signal sensor module 1a further includes a cushioning material 42. The body motion sensor 12 (e.g., a second sensor) is stacked on the biological signal sensor 11 (e.g., a first sensor) with the cushioning material 42 interposed between them. The adhesive material 41 is disposed on a first main surface 33 of the biological signal sensor 11. The cushioning material 42 is disposed on a second main surface 34 on the opposite side to the first main surface 33 in the biological signal sensor 11.

As further illustrated in FIG. 8, the biological signal sensor module 1a does not include the low-gain circuit 14. Outputs of the biological signal sensor 11 and the body motion sensor 12 are input to the limiter circuit 13. The limiter circuit 13 amplifies output of the biological signal sensor 11 and output of the body motion sensor 12 with the same gain.

It is also noted that the limiter circuit 13 is configured to separately handle output of the biological signal sensor 11 and output of the body motion sensor 12.

As described above, the limiter circuit 13 amplifies output of the biological signal sensor 11 so as to detect deformation of a skin surface due to a pulse wave and body motion and output a mixed signal.

Furthermore, the body motion sensor 12 is attached to a skin surface with the cushioning material 42 interposed between them. For this reason, displacement of a skin surface is less likely to be transmitted to the body motion sensor 12. By this configuration, as compared with output of the biological signal sensor 11 according to displacement of a skin surface by a pulse wave, output of the body motion sensor 12 according to displacement of a skin surface by body motion is substantially the same, and output of the body motion sensor 12 according to displacement of a skin surface by a pulse wave is very small. On the other hand, as described above, the limiter circuit 13 amplifies output of the biological signal sensor 11 and output of the body motion sensor 12 with the same gain. Therefore, the limiter circuit 13 amplifies output of the body motion sensor 12 so as to detect deformation of a skin surface due to body motion and output a body motion signal.

According to the present modification example, since a low-gain circuit is not required to obtain a body motion signal, a simpler biological signal sensor module can be realized.

### Third modification example

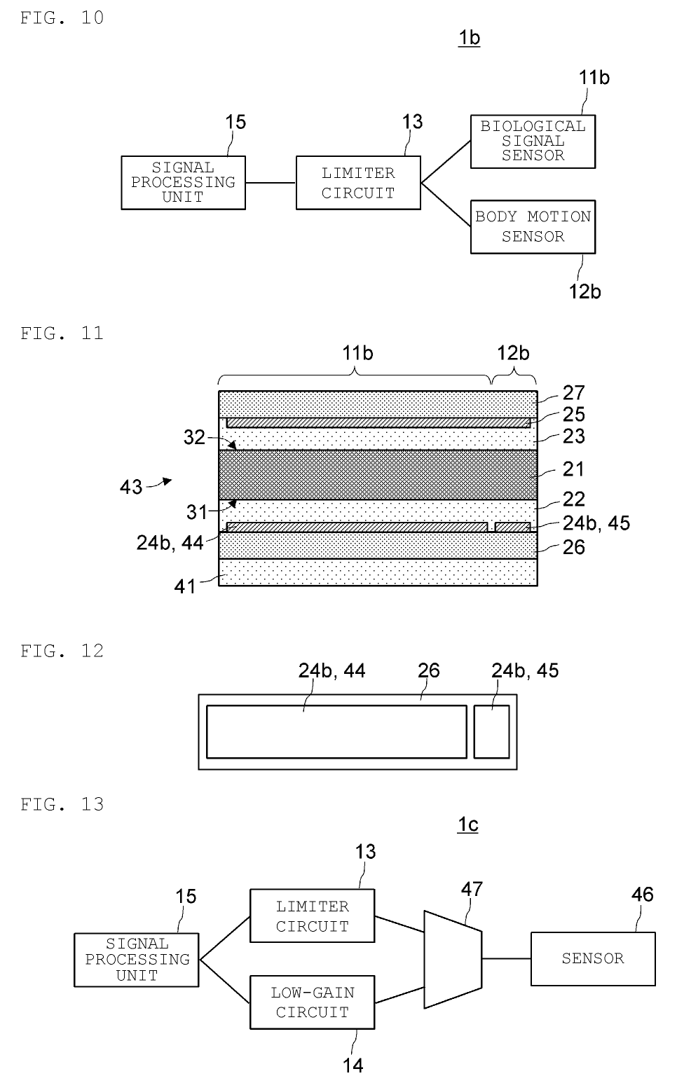

FIG. 10 is a block diagram illustrating a biological signal sensor module 1b. FIG. 11 is a sectional view illustrating a biological signal sensor 11b and a body motion sensor 12b. FIG. 12 is a plan view illustrating a signal electrode 24b. The biological signal sensor module 1b is different from the biological signal sensor module 1 in the following point. That is, as illustrated in FIG. 11, the biological signal sensor module 1b includes the integrated biological signal sensor 11b (e.g., a first sensor) and body motion sensor 12b (e.g., a second sensor). As illustrated in FIG. 10, the biological signal sensor module 1b does not include the low-gain circuit 14. Outputs of the biological signal sensor 11b and the body motion sensor 12b are input to the limiter circuit 13. The limiter circuit 13 amplifies the output of the biological signal sensor 11b and the output of the body motion sensor 12b with the same gain.

More specifically, as illustrated in FIGS. 11 and 12, the biological signal sensor module 1b includes a laminate 43. The biological signal sensor 11b and the body motion sensor 12b are formed in the laminate 43. The laminate 43 includes the piezoelectric film 21, the adhesive materials 22 and 23, a signal electrode 24b, the reference electrode 25, and the flexible substrates 26 and 27. The signal electrode 24b is formed on the flexible substrate 26, and is attached to the first main surface 31 of the piezoelectric film 21 by the adhesive material 22. Further, the signal electrode 24b includes a first electrode 44 and a second electrode 45 having an area smaller than an area of the first electrode 44 in plan view of the first main surface 31 of the piezoelectric film 21. The reference electrode 25 is formed on the flexible substrate 27 and is attached to the second main surface 32 of the piezoelectric film 21 by the adhesive material 23. The adhesive material 41 is disposed on a main surface of the laminate 43.

According to this exemplary aspect, the biological signal sensor 11b corresponds to a portion overlapping the first electrode 44 in the laminate 43 when viewed from a laminating direction of the laminate 43, and includes the first electrode 44 disposed on the first main surface 31 of the piezoelectric film 21. The body motion sensor 12b corresponds to a portion overlapping the second electrode 45 of the laminate 43 when viewed from the laminating direction of the laminate 43 and includes the second electrode 45 disposed on the first main surface 31 of the piezoelectric film 21.

As described above, in plan view of the first main surface 31 of the piezoelectric film 21, the second electrode 45 has an area smaller than an area of the first electrode 44. For this reason, the body motion sensor 12b has output smaller than output of the biological signal sensor 11b with respect to the same displacement of a skin surface. By this configuration, as compared with output of the biological signal sensor 11b according to displacement of a skin surface by a pulse wave, output of the body motion sensor 12b according to displacement of a skin surface by body motion is substantially the same, and output of the body motion sensor 12b according to displacement of a skin surface by a pulse wave is very small. Therefore, similar to the case of the biological signal sensor module 1a, the limiter circuit 13 amplifies output of the biological signal sensor 11b so as to detect deformation of a skin surface due to a pulse wave and body motion and output a mixed signal. Furthermore, the limiter circuit 13 amplifies output of the body motion sensor 12b so as to detect deformation of a skin surface due to body motion and output a body motion signal.

According to the present modification example, since a low-gain circuit is not required to obtain a body motion signal, a simpler biological signal sensor module can be realized.

### Fourth modification example

FIG. 13 is a block diagram illustrating a biological signal sensor module 1c. The biological signal sensor module 1c is different from the biological signal sensor module 1 in the following point. That is, the biological signal sensor module 1c includes one sensor 46 and one demultiplexer 47.

The sensor 46 has the same structure as the biological signal sensor 11, and the adhesive material 41 is disposed in the same manner as the biological signal sensor 11. The sensor 46 outputs a signal due to deformation of a skin surface. The demultiplexer 47 selects either the limiter circuit 13 (e.g., a first detection circuit) or the low-gain circuit 14 (e.g., a second detection circuit), and outputs a signal from the sensor 46. More specifically, the demultiplexer 47 continuously switches an output destination of a signal from the sensor 46 at a high speed, and allocates a part (e.g., a first output) of the signal from the sensor 46 to the limiter circuit 13 and routes the rest (e.g., a second output) of the signal from the sensor 46 to the low-gain circuit 14. As a result, a mixed signal output from the limiter circuit 13 and a body motion signal output from the low-gain circuit 14 can be regarded as reflecting deformation of a skin surface at the same time.

According to the present modification example, since a plurality of sensors are not required to obtain a mixed signal and a body motion signal, a simpler biological signal sensor module can be realized.

### Additional Modification examples

The biological signal sensor module can be configured to estimate a biological signal according to deformation of a skin surface due to tremor that occurs involuntarily when muscles contract periodically.

Moreover, the biological signal sensor module can be configured to estimate a biological signal corresponding to vibration of a skin surface generated in a state where a fingertip touches an object.

The biological signal sensor module can also be configured to estimate a biological signal corresponding to deformation derived from a blood vessel, deformation derived from external force, or deformation when a person moves his/her body.

The biological signal sensor module is particularly advantageous for estimation of periodic biological signals, such as pulse wave signals.

It should be appreciated that the configuration described in each of the above embodiments may be appropriately replaced or combined.

### DESCRIPTION OF REFERENCE SYMBOLS

1, 1a to 1c: Biological signal sensor module
11, 11b: Biological signal sensor
12, 12b: Body motion sensor
13: Limiter circuit
14: Low-gain circuit
15: Signal processing unit
21: Piezoelectric film
22, 23, 41: Adhesive material
24, 24b: Signal electrode
25: Reference electrode
26, 27: Flexible substrate
42: Cushioning material
43: Laminate
44: First electrode
45: Second electrode
46: Sensor
47: Demultiplexer

1. A biological signal sensor module comprising:
at least one sensor that has a piezoelectric film and outputs a first output and a second output based on a deformation of a skin surface;
at least one detection circuit that amplifies the first output to output a first signal that includes a biological signal and a body motion signal, and further amplifies the second output to output a second signal that includes the body motion signal; and
a signal processing unit configured to estimate a biological signal based on the first signal and the second signal by using a preset biological signal estimation algorithm.

2. The biological signal sensor module according to claim 1, wherein the biological signal estimation algorithm is a neural network set by machine learning using the first signal, the second signal, and the biological signal as training data.

3. The biological signal sensor module according to claim 2, wherein the biological signal estimation algorithm is an LSTM neural network.

4. The biological signal sensor module according to claim 2, wherein the biological signal estimation algorithm is a bidirectional LSTM neural network.

5. The biological signal sensor module according to claim 1, further comprising a cushioning material.

6. The biological signal sensor module according to claim 5, wherein the at least one sensor includes:
a first sensor that outputs the first output; and
a second sensor that outputs the second output, and
wherein the second sensor is laminated on the first sensor with the cushioning material interposed therebetween.

7. The biological signal sensor module according to claim 6, wherein the at least one detection circuit is a single detection, amplifies the first output and the second output with a same gain as each other.

8. The biological signal sensor module according to claim 1, wherein:
the at least one sensor includes a first sensor that outputs the first output, and a second sensor that outputs the second output,
the piezoelectric film has a first main surface and a second main surface that opposes the first main surface, and
the first sensor includes a first electrode disposed on the first main surface of the piezoelectric film.

9. The biological signal sensor module according to claim 8, wherein the second sensor includes a second electrode disposed on the first main surface of the piezoelectric film and having an area smaller than an area of the first electrode in a plan view of the first main surface of the piezoelectric film.

10. The biological signal sensor module according to claim 9, wherein the at least one detection circuit is a single detection, amplifies the first output and the second output with a same gain as each other.

11. The biological signal sensor module according to claim 1, wherein:
the at least one sensor is a single sensor, and
the at least one detection circuit includes a first detection circuit that outputs the first signal and a second detection circuit that outputs the second signal.

12. The biological signal sensor module according to claim 11, further comprising a demultiplexer that routes the first output to the first detection circuit and the second output to the second detection circuit.

13. The biological signal sensor module according to claim 1, wherein the at least one detection circuit includes:
a limiter circuit that amplifies the first output to output the first signal that includes the biological signal and the body motion signal, and
a low-gain circuit that amplifies the second output to output the second signal that includes the body motion signal.

14. The biological signal sensor module according to claim 13, wherein the limiter circuit is adjusted in gain so as to amplify a displacement of the skin surface due to a pulse wave accompanying a heartbeat, and the limiter circuit further decreases gain with respect to the first signal corresponding to a displacement of the skin surface due to body motion.

15. The biological signal sensor module according to claim 14, wherein the low-gain circuit is adjusted in gain to amplify the second signal corresponding to the displacement of the skin surface due to body motion.

16. The biological signal sensor module according to claim 13, wherein the piezoelectric film of the at least one sensor is configured to deform in a thickness direction in response to the deformation of the skin surface of the user and to generate a polarization charge.

17. The biological signal sensor module according to claim 16, wherein the limiter circuit and the low-gain circuit are configured to convert the polarization charge into a voltage and amplify the voltage with a predetermined gain.

18. The biological signal sensor module according to claim 1, wherein the at least one sensor comprises a biological signal sensor and a body motion sensor that are formed in a laminate.

19. The biological signal sensor module according to claim 18, wherein the laminate includes the piezoelectric film, a signal electrode, a reference electrode, a first flexible substrate, and a second flexible substrate.

20. The biological signal sensor module according to claim 19, wherein:
the signal electrode is disposed on the first flexible substrate and is attached to a first main surface of the piezoelectric film,
the signal electrode includes a first electrode and a second electrode having an area smaller than an area of the first electrode in a plan view of the first main surface of the piezoelectric film, and
the reference electrode is disposed on the second flexible substrate and is attached to a second main surface of the piezoelectric film.
